(19) United States
(12) Patent Application Publication
(10) Pub. No.: US 2026/0174401 A1
(43) Pub. Date: Jun. 25, 2026

(54) CAPTURE AND DISPLAY OF HEALTH INFORMATION

(71) Applicant: Welch Allyn, Inc., Skaneateles Falls, NY (US)

(72) Inventors: WonKyung McSweeney, Manlius, NY (US); Danielle R. Endres, Syracuse, NY (US); Corinn C. Fahrenkrug, Chittenango, NY (US); Michael Holtz, Rochester, NY (US); Catherine M. King, Portland, OR (US); John A. Lane, Venice, FL (US); Craig M. Meyerson, Syracuse, NY (US); Thomas A. Myers, Syracuse, NY (US); Anzhelika Polshikova, Syracuse, NY (US); David E. Quinn, Moravia, NY (US); Nancy Tran, Syracuse, NY (US); Gene J. Wolfe, Pittsford, NY (US)

(21) Appl. No.: 19/414,769

(22) Filed: Dec. 10, 2025

Related U.S. Application Data
(60) Provisional application No. 63/737,005, filed on Dec. 20, 2024.

(51) Int. Cl. A61B 5/00; G06F 21/84; G16H 10/60; G16H 40/60

(52) U.S. Cl. CPC A61B 5/742; A61B 5/749; G06F 21/84; G16H 10/60; G16H 40/60

(57) ABSTRACT

A system for capturing and displaying health information. The system receives data from one or more devices monitoring a patient, detects an event based on the data received from the one or more devices, and generates an instruction for displaying the event. The instruction includes an identification of one or more authorized roles for viewing the event and an identification of one or more authorized locations for displaying the event. The system determines a location of the display device and detects a user within a threshold distance of the display device. The system displays the event on the display screen when the location of the display device is included in the one or more authorized locations for displaying the event and a role of the user within the threshold distance of the display device is included in the one or more authorized roles for viewing the event.

### CROSS-REFERENCE TO RELATED APPLICATION

This application claims the benefit of U.S. Provisional Application No. 63/737,005, filed Dec. 20, 2024, the disclosure of which is hereby incorporated by reference in its entirety.

### BACKGROUND

In medical-surgical nursing, a common challenge is difficulty in accessing critical patient information at the bedside where it is often needed most. Nurses often juggle multiple patients and need up-to-date details on medications, lab results, and care plans to provide safe and effective care. However, this information can be scattered across different platforms or buried in complex electronic health records (EHRs), leading to delays and potential errors. The lack of immediate access to comprehensive patient data can hinder decision-making, slow down workflows, and increase stress for nurses, which can ultimately impact patient outcomes.

### SUMMARY

In general terms, the present disclosure relates to capturing and displaying health information. In one possible configuration, an event is displayed based on a location of a display device and a role of a user within a threshold distance of the display device. In another possible configuration, a first set of health information is displayed based on a first location of a display screen, a second set of health information is displayed based on a second location of the display screen, and the second set of health information has a specificity different from a specificity of the first set of health information. Various aspects are described in this disclosure, which include, but are not limited to, the following aspects.

One aspect relates to a system for capturing and displaying health information, the system comprising: a display device including: a housing; a display screen supported on the housing; at least one processing device communicatively connected to the display screen; and at least one memory device storing software instructions that, when executed by the at least one processing device, cause the at least one processing device to: receive data from one or more devices monitoring a patient; detect an event based on the data received from the one or more devices; generate an instruction for displaying the event, the instruction including an identification of one or more authorized roles for viewing the event and an identification of one or more authorized locations for displaying the event; determine a location of the display device; detect a user within a threshold distance of the display device; and display the event on the display screen when the location of the display device is included in the one or more authorized locations for displaying the event and a role of the user within the threshold distance of the display device is included in the one or more authorized roles for viewing the event.

Another aspect relates to a method of dynamically displaying health information, the method comprising: detecting a first location of a display screen; displaying a first set of health information on the display screen based on the first location, the first set of health information having a first specificity; detecting a second location of the display screen, wherein the second location of the display screen differs from the first location of the display screen; and displaying a second set of health information on the display screen based on the second location, the second set of health information having a second specificity, and wherein the second specificity differs from the first specificity.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system for capturing and displaying health information, the system including a patient support apparatus and a display device mounted on the patient support apparatus.

FIG. 2 schematically illustrates an example of the display device of FIG. 1.

FIG. 3 schematically illustrates an example of a method of capturing and displaying health information that can be performed by the system of FIG. 1.

FIG. 4 illustrates an example of a graphical user interface displayed on the display device of FIG. 1 in accordance with the method of FIG. 3.

FIG. 5 illustrates another example of a graphical user interface displayed on the display device of FIG. 1 in accordance with the method of FIG. 3.

FIG. 6 illustrates another example of a graphical user interface displayed on the display device of FIG. 1 in accordance with the method of FIG. 3.

FIG. 7 schematically illustrates an example of a data structure for displaying an event on the display device in accordance with the operations of the method of FIG. 3.

FIG. 8 schematically illustrates an example of data entries that can be generated by the display device of FIG. 1 based on inputs and/or commands received from an authorized user.

FIG. 9 schematically illustrates an example of a method of dynamically displaying health information that can be performed on the display device of FIG. 1, or alternatively, on a mobile device carried by a healthcare professional in a healthcare facility.

### DETAILED DESCRIPTION

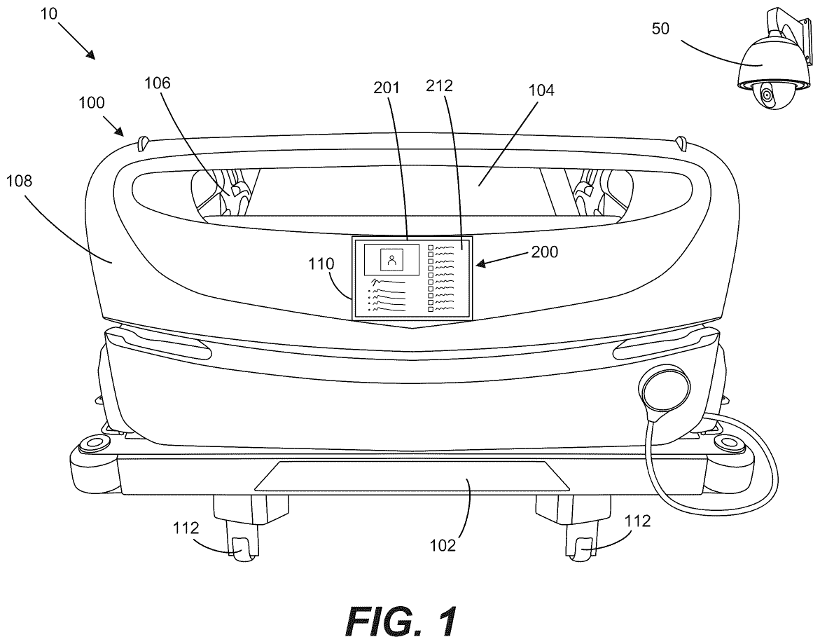

FIG. 1 illustrates an example of a system 10 for capturing and displaying health information. As shown in FIG. 1, the system 10 includes a patient support apparatus 100 and a display device 200 mounted on the patient support apparatus 100. As will be described in more detail, the display device 200 displays an event based on a location of the display device 200 and a role of a user within a threshold distance of the display device 200.

In the example illustrated in FIG. 1, the patient support apparatus 100 is shown as a hospital bed. In other examples, the patient support apparatus 100 can include a stretcher, surgical table, or other structure configured to support a patient within a healthcare environment such as a hospital, nursing home, long term care facility, and the like.

The patient support apparatus 100 includes a frame 102 that supports a mattress 104. The frame 102 can include one or more electronic motors to raise and lower the mattress 104 relative to the ground, and to adjust angles between one or more sections of the mattress 104 such as angles between head, thigh, and foot sections of the mattress 104. The frame 102 further includes casters 112 to facility transporting the patient support apparatus 100 around the medical facility such as from one patient room to another patient room.

The patient support apparatus 100 can further include one or more sensors that capture measurements of one or more physiological variables of a patient resting on the mattress 104 such as heart rate, respiration rate, weight, and motion. For example, the one or more sensors can include load cells that detect the weight and center of gravity of the patient while resting on the mattress 104, and can detect motion of the patient such as when the patient turns from one side to another, or when the patient exists the patient support apparatus 100. The one or more sensors can be positioned under the mattress 104, can be embedded in the mattress 104, can be positioned on top of the mattress 104, or can be positioned elsewhere on the frame 102.

The patient support apparatus 100 can further include one or more railings that when deployed prevent the patient from exiting the patient support apparatus from one or more sides. In the example of FIG. 1, the patient support apparatus 100 is shown as having a left side rail 106 in a deployed position preventing the patient from exiting the patient support apparatus 100 on the left side, while the patient support apparatus 100 has a right side rail in a stowed position allowing the patient to exit the patient support apparatus 100 on the right side. The patient support apparatus 100 further includes a footboard 108 preventing the patient from exiting the patient support apparatus 100 at the foot section of the patient support apparatus 100.

As further shown in FIG. 1, the system 10 further includes a camera 50 that captures visual data of the patient such as while resting on the patient support apparatus 100, or when the patient exits the patient support apparatus 100. The visual data can include images, videos, and the like. The visual data captured by the camera 50 can be analyzed to detect clinically relevant events such as when the patient is turned on the patient support apparatus 100, when the patient exits the patient support apparatus 100 such as to toilet, when the patient intakes food or fluids, when wound care is provided to the patient, and other clinically relevant events.

As shown in FIG. 1, the display device 200 is mounted on the footboard 108 of the patient support apparatus 100. In alternative examples, the display device 200 can be mounted on the left or right siderails, or elsewhere on the patient support apparatus 100. In yet further examples, the display device 200 can be mounted elsewhere in the room where the patient support apparatus 100 is located such as on a wall, a fixture, or on another medical device.

The footboard 108 includes a docking station 110 for mounting the display device 200 onto the frame 102 of the patient support apparatus 100. When mounted in the docking station 110, the display device 200 is physically attached to the patient support apparatus 100. The display device 200 has a housing 201 that supports a display screen 212. The housing 201 is removably attachable to the docking station 110 of the patient support apparatus 100. Accordingly, the display device 200 is detachable from the patient support apparatus 100 such that the display device 200 can be moved by a user such as a clinician to any area around the patient support apparatus 100 to better use its features and dynamic display. In alternative examples, the docking station 110 can be separate from the patient support apparatus 100. In yet further examples, the system 10 does not include a docking station 110.

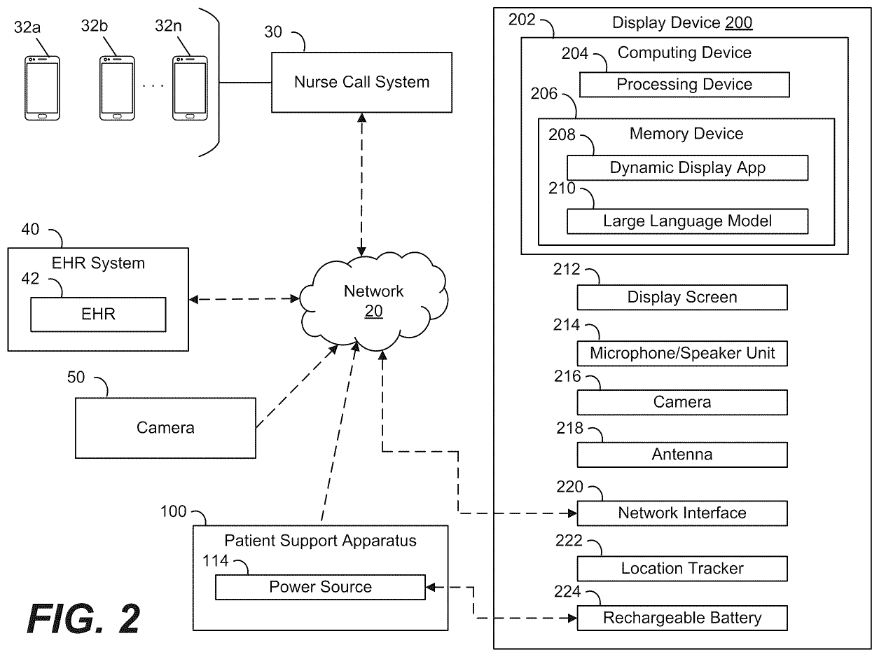

The display device 200 can include a rechargeable battery 224 (see FIG. 2) that is recharged when the display device 200 is mounted in the docking station 110. For example, the docking station 110 can include a power source 114 (see FIG. 2) that wirelessly recharges the rechargeable battery 224 of the display device 200 via inductive charging (also known as wireless charging or cordless charging) which is a type of wireless power transfer that uses electromagnetic induction. Alternatively, the housing 201 can include electrical contacts that contact corresponding electrical contacts inside the docking station 110 to form an electrical connection that can transfer power from the power source 114 of the patient support apparatus 100 for recharging the rechargeable battery 224 of the display device 200.

The display device 200 can communicate with the patient support apparatus 100 through an electrical connection established through the docking station 110. For example, the electrical connection formed between the electrical contacts on the housing 201 of the display device 200 and the corresponding electrical contacts inside the docking station 110 can be used to transfer both power and data between the patient support apparatus 100 and the display device 200. Alternatively, the display device 200 can communicate with the patient support apparatus 100 wirelessly such as through a network 20 (see FIG. 2), or through a direct wireless connection established through Wi-Fi, Ultra-wideband (UWB), Bluetooth, and other wireless technologies.

FIG. 2 schematically illustrates an example of the display device 200 communicatively connected via a network 20 to a nurse call system 30, an electronic health record (EHR) system 40, the camera 50, and the patient support apparatus 100. Alternatively, the display device 200 can directly connect (i.e., without using the network 20) to one or more of the nurse call system 30, the EHR system 40, the camera 50, and the patient support apparatus 100.

In the example embodiment shown in FIG. 2, the display device 200 is connected to EHR system 40 and to other devices that capture data such as the camera 50 and the patient support apparatus 100. The display device 200 detects an identity of the patient, and based on which user is detected nearby, the display device 200 curates a unique set of information for display. For example, when a registered nurse is detected as being within a threshold distance of the display device 200, a task list that is specific to the role of the registered nurse is displayed. Also, events since the registered nurse's last visit to the patient that are detected by the display device 200 or the other devices in communication with the display device 200 can be displayed. Further, relevant vital signs information specific to the needs and condition of the patient can be displayed on the display device 200. Information that is relevant to the current registered nurse is displayed on the display device 200, while it is not displayed for other registered nurses. Thus, the information displayed on the display device 200 may vary from other users including other registered nurses, clinicians, physical therapists, technicians, and the like.

In another example embodiment, the display device 200 does not connect to EHR system 40 continuously, but only to one or more devices that capture data such as the camera 50 and the patient support apparatus 100. In such example embodiment, the display devices 200 acts as a hands-free interface for a clinician's mobile device and its features. The display device 200 can be configured to load relevant information for the patient that the clinician is interacting with and provide hands-free interactions optimized just for the patient. The display device 200 may be used to update an EHR 42 of a patient with its stored data whenever the clinician chooses to connect to the EHR system 40. The update can be two-way, but clinician can choose which information should go to EHR 42. The display device 200 can update the display screen 212 with data retrieved from the EHR 42 of the patient and then disconnect from the EHR system 40.

As shown in FIG. 2, the display device 200 includes a computing device 202 having at least one processing device 204 and at least one memory device 206 that stores software instructions that, when executed by the at least one processing device 204, cause the at least one processing device 204 to perform the various aspects, functions, and operations described herein.

The at least one processing device 204 is an example of a processing unit such as a central processing unit (CPU). The at least one processing device 204 can include one or more CPUs. In some examples, the at least one processing device 204 includes one or more digital signal processors, field-programmable gate arrays, and/or other types of electronic circuits.

The at least one memory device 206 is an example of a computer-readable data storage device that operates to store data and instructions for execution by the at least one processing device 204. As shown in FIG. 2, the at least one memory device 206 stores a dynamic display application 208 that generates an instruction for displaying an event detected based on data received from one or more devices monitoring a patient assigned to the patient support apparatus 100. The instruction includes an identification of one or more authorized roles for viewing the event and an identification of one or more authorized locations for displaying the event. The dynamic display application 208 displays the event on the display screen 212 when the location of the display device 200 is included in the one or more authorized locations for displaying the event and a role of a user within a threshold distance of the display device 200 is included in the one or more authorized roles for viewing the event.

The at least one memory device 206 further stores a large language model (LLM) 210, which is a type of artificial intelligence (AI) program that uses deep learning to analyze speech such as during interactions between a user within a threshold distance of the display device 200 and the patient assigned to the patient support apparatus 100. For example, the LLM 210 can generate an event based on the analysis of the speech for displaying the event on the display screen 212 of the display device 200. Upon confirmation from the user within the threshold distance of the display device 200, the event can be stored locally on the display device, or can be stored in an external system such as the EHR system 40, or can be discarded.

The at least one memory device 206 includes computer-readable media, which includes any media that can be accessed by the at least one processing device 204. The computer-readable media can include computer-readable storage media and computer-readable communication media. The computer-readable storage media includes volatile and nonvolatile, removable and non-removable media implemented in any device that can store information such as computer-readable instructions, data structures, program modules, or other data. The computer-readable storage media can include random access memory, read only memory, electrically erasable programmable read only memory, flash memory, and other memory technology, including any medium that can be used to store information that can be accessed by the at least one processing device 204. The computer-readable storage media is non-transitory.

The computer-readable communication media embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. The computer-readable communication media can include wired media such as a wired network or direct-wired connection, and wireless media such as acoustic, radio frequency, infrared, and other wireless media. Combinations of any of the above are within the scope of computer-readable media.

As shown in FIG. 2, the display device 200 includes the display screen 212 which is a touch sensitive touchscreen that receives inputs from authorized users such as physicians, registered nurses (RN), licensed practical nurses (LPN), or other healthcare professionals. The display screen 212 can use various technologies to sense the inputs such as capacitance to detect changes in an electrical field when a conductive object (e.g., finger or stylus) touches the screen. When the display screen 212 detects an input, the display screen 212 sends data to the at least one processing device 204, which interprets the input and responds accordingly.

In some examples, the display screen 212 is configured to detect touch and movement of a stylus such as a pen that is compatible with the display screen 212. In such examples, authorized users of the display device 200 can each have their own stylus that they carry around with them in the healthcare facility to prevent contamination when the display device 200 is used by multiple users. Additionally, each stylus can emit a unique signal that acts as a locator within the healthcare facility for each authorized user of the display device 200. Further, each stylus can also be used as an authentication token associated with the authorized user. Other physical objects can also be used as authentication tokens for the authorized user including wireless tags that can be attached to objects worn by the user such as lanyards, name tags, and the like.

In some examples, the display device 200 can further include a sensor that detects gestures from an authorized user within a threshold distance of the display device 200. The gestures detected by the display device 200 are analyzed to determine inputs for performing one or more actions on the display device 200 without the user having to physically touch the display screen 212. For example, the display device 200 can detect a hand gesture from an authorized user to confirm one or more actions for an event detected by the display device 200 such as to chart the event for storage in an electronic health record (EHR) 42 managed by the EHR system 40, to store the event as a non-chartable event locally on the display device 200 or elsewhere, or to message the event to another authorized user by using the nurse call system 30.

The display device 200 includes a microphone/speaker unit 214 that detects sounds around the display device 200. For example, when the display device 200 is mounted on the patient support apparatus 100, the microphone/speaker unit 214 can perform ambient listening to detect sounds of the patient while resting on the patient support apparatus 100, and to monitor and/or record conversations between users within a threshold distance of the display device 200 and the patient. The sounds detected by the microphone/speaker unit 214 can be analyzed by the LLM 210 to detect an event which can be displayed on the display screen 212 when an authorized user is detected to be within the threshold distance of the display device 200.

The speech detected by the microphone/speaker unit 214 can be analyzed by the LLM 210 to determine voice commands from the user within the threshold distance of the display device 200 such as voice commands to add more information and/or notes, and to capture vital sign measurements acquired from other devices such as the patient support apparatus 100. The LLM 210 can also generate outputs for the microphone/speaker unit 214 such as audio instructions for one or more tasks that need to be done by bedside (e.g., “Make sure to tell the physical therapist to check for XYZ”), as well as initiating requests or providing communication updates via phones, email, or other linked communication systems.

The display device 200 can include a camera 216 that can be used to capture images or video of the patient, or of an environment around the patient. As an illustrative example, when the display device 200 is removed from the docking station 110 the camera 216 can be used to take an image of a setup, a wound, or other items that can be displayed on the display screen 212 during a subsequent visit to the patient such as to jog a memory of an authorized user such as a registered nurse. Additionally, the images and videos captured by the camera 216 can be charted and saved to the EHR 42 of the patient that is managed by the EHR system 40.

The display device 200 can include an antenna 218 to identify authorized users of the display device 200 via proximity detection technology. The antenna 218 can be used to detect wireless signals from objects worn or carried by the authorized users of the display device 200. The wireless signals detected by the antenna 218 can include radio-frequency identification (RFID) signals, near-field communication (NFC) signals, Wi-Fi signals, and/or Bluetooth® signals. The wireless signal from each object is unique for uniquely identifying each authorized user. Further, the antenna 218 can be used to detect whether the authorized user is within the threshold distance of the display device 200 for dynamically updating the display screen 212.

In further examples, identification of the authorized users of the display device 200 can be accomplished by analyzing the visual data captured by the camera 50 (see also FIG. 1). For example, facial recognition can be performed on the visual data captured by the camera 50 to determine an identity of a user within the threshold distance of the display device 200. Alternatively, or additionally, the visual data captured by the camera 50 can be analyzed to detect machine-readable data, a name, or other identification on an object worn by the user such as a badge, lanyard, name tag, and the like to determine the identity of the user.

The display device 200 includes a network interface 220 that allows the display device 200 to connect to the network 20. The network interface 220 can include wired interfaces and/or wireless interfaces. For example, the network interface 220 can wirelessly connect to the network 20 such as through Wi-Fi and other wireless communications protocols. Alternatively, or additionally, the network interface 220 can connect to the network 20 using wired connections such as through Ethernet or Universal Serial Bus (USB) cables.

The network 20 can include any type of wired or wireless connections or any combinations thereof. Examples of wireless connections include Wi-Fi, Bluetooth, ultra-wideband (UWB), radio frequency identification (RFID), cellular network connections, and the like. In some examples, the network 20 is an Internet-of-things (IoT) network that connects and exchanges data between one or more devices and the display device 200 over the Internet or other communications networks. In alternative examples, one or more of the devices can directly communicate with the display device 200 without using the network 20 such as via direct wireless or wired connections between the one or more devices and the display device 200.

The display device 200 can include a location tracker 222 that can track a location of the display device 200. The location tracker 222 can utilize Bluetooth Low Energy (BLE) to broadcast a Bluetooth signal that is detected by other devices connected to the network 20 that are proximate to the display device 200. The location of the display device 200 is determined based on the location of the other devices that are connected to the network 20. In further examples, the location tracker 222 can utilize global positioning system (GPS) technology to track the location of the display device 200. Use of additional technologies for determining the location of the display device 200 is contemplated.

The location tracker 222 can be used to find the display device 200 such as when the display device 200 is misplaced or lost within the healthcare facility. In some examples, an alarm is triggered when the display device 200 is moved beyond a threshold distance away from a designated location such as a patient room within the healthcare facility. In some examples, the alarm is triggered when the display device 200 is moved beyond a threshold distance away from the patient support apparatus 100 such as when the Bluetooth signal from the location tracker 222 is no longer detected by the patient support apparatus 100.

In further examples, the alarm is triggered when the display device 200 is moved beyond a threshold distance away from an authorized user and/or the patient when display device 200 is removed from the docking station 110. In some examples, the docking station 110 is locked such that the docking station 110 prevents removal of the display device 200 when an authorized user is not within a threshold distance of the patient support apparatus 100.

In some instances, the patient can exit the patient support apparatus 100 to travel for a test, a surgery, or other type of procedure, and the display device 200 travels with the patient. In such cases, when the display device 200 stays with the patient, the alarm is not triggered.

When the alarm is triggered, the alarm can be communicated to one or more mobile devices 32 via the nurse call system 30. In some instances, the alarm is triggered on the display device 200 such as a visual alarm displayed on the display screen 212 or an audible alarm emitted by the microphone/speaker unit 214. In some examples, the display device 200 is frozen to prevent unauthorized use when the display device 200 is moved beyond the threshold distance away from the designated area or the patient support apparatus 100.

In the example shown in FIG. 2, the display device 200 is a standalone device with all necessary hardware such as the display screen 212, the microphone/speaker unit 214, the camera 216, and the antenna 218. In alternative examples, the aspects of the system 10 can be achieved through distributed devices that are connected to the network 20 through a specific protocol.

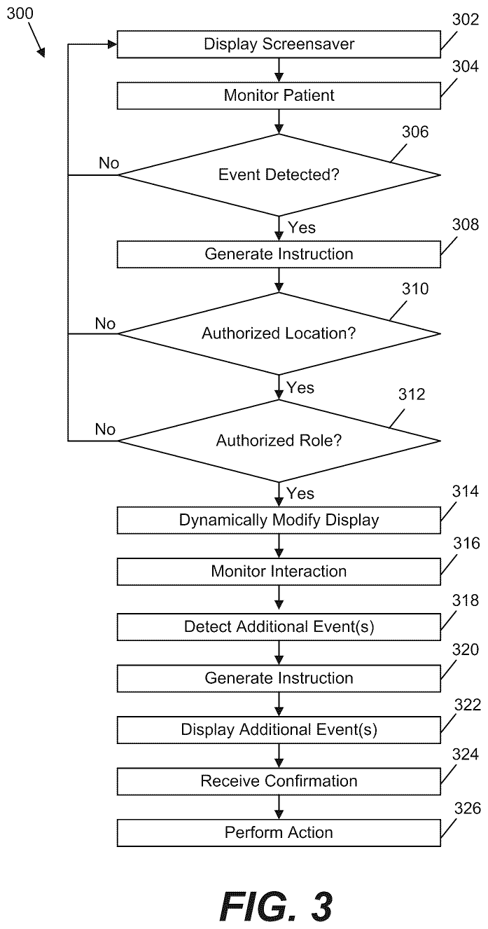

FIG. 3 schematically illustrates an example of a method 300 of capturing and displaying health information that can be performed by the system 10. At least some of the aspects of the method 300 are performed by the display device 200.

The method 300 includes an operation 302 of displaying a screensaver on the display screen 212 of the display device 200. The screensaver blocks protected health information and other confidential information from being displayed on the display screen 212.

Operation 302 is performed when the location of the display device 200 is not in one or more authorized locations or when a user having an authorized role is not detected within a threshold distance of the display device 200. As described above, the location of the display device 200 can be determined from the location tracker 222 broadcasting the BLE signal that is detected by other devices connected to the network 20. Also, detection of the authorized user can be determined by the antenna 218 detecting wireless signals from objects worn or carried by the authorized user, or from analysis of the visual data captured by the camera 50.

The method 300 includes an operation 304 of receiving data from one or more devices monitoring the patient assigned to the patient support apparatus 100. Operations 302, 304 can occur simultaneously such that the screensaver is displayed on the display screen 212 while the display device 200 receives the data monitoring the patient.

The data received from the one or more devices in operation 304 can include data from one or more sensors that capture measurements of one or more physiological variables of the patient such as the one or more sensors of the patient support apparatus 100 that capture measurements of heart rate, respiration rate, weight, and motion. Clinically relevant events can be derived from the motion detected by the one or more sensors of the patient support apparatus 100 such as when the patient turns or exits the patient support apparatus 100.

In further examples, the data received from the one or more devices in operation 304 can include the visual data captured by the camera 50. The visual data captured by the camera 50 can includes images and/or videos of clinically relevant events such as when the patient turns on the patient support apparatus 100, when the patient exits the patient support apparatus 100 such as to toilet, and when the patient intakes food and/or beverages.

The method 300 includes an operation 306 of determining whether an event is detected based on the data received from the one or more devices in operation 304. Operation 306 can include analyzing the visual data captured by the camera 50 to detect an event such as when the patient turns on the patient support apparatus 100, exits the patient support apparatus 100 to toilet, or intakes food and/or beverages. Operation 306 can also include analyzing measurements of one or more physiological variables captured by the one or more sensors on the patient support apparatus 100 to detect an event such as a physiological variable measurement that is outside of a normal range, a level of activity of the patient, and the like.

As illustrated in FIG. 3, when an event is not detected based on the data received from the one or more devices (i.e., “No” in operation 306), the method 300 returns to operation 302 of displaying the screensaver and operation 304 of monitoring the patient, which as described above, can occur simultaneously. When an event is detected based on the data received from the one or more devices (i.e., “Yes” in operation 306), the method 300 proceeds to an operation 308 of generating an instruction for displaying the event on the display screen 212.

The instruction generated in operation 308 includes an identification of one or more authorized roles for viewing the event. Users of the display device 200 may have different roles such as physicians, registered nurses (RN), licensed practical nurses (LPN), and other healthcare professionals. Each of these roles may have different authorizations for viewing events on the display device 200 such that some roles are authorized to view an event (i.e., physicians and registered nurses) while other roles are not authorized (i.e., licensed practical nurses).

The instruction also includes an identification of one or more authorized locations for displaying the event. For example, the event can be displayed on the display device 200 in a first location such as a patient room in the healthcare facility while the event is blocked from being displayed in a second location such as a hallway in the healthcare facility.

As discussed above, the display device 200 can be mounted on the docking station 110 of the patient support apparatus 100, and the patient support apparatus 100 is portable. Thus, when docked on the patient support apparatus 100, the display device 200 dynamically updates the display screen 212 based on the location of the patient support apparatus 100. In further examples, when the display device 200 is removed from the docking station 110 such that the display device 200 is no longer attached to the patient support apparatus 100, the display device 200 dynamically updates the display screen 212 based on the location of the display device 200 independent of the location of the patient support apparatus 100.

The method 300 includes an operation 310 of determining whether a location of the display device 200 is an authorized location defined by the instruction generated in operation 308. Operation 310 can include determining a location of the display device 200 in accordance with the examples described above. For example, operation 310 can include using the location tracker 222 to determine the location of the display device 200. Once the location of the display device 200 is determined, operation 310 can then include determining whether the location of the display device 200 is included in one or more authorized locations defined by the instruction.

As illustrated in FIG. 3, when the location of the display device 200 is not an authorized location defined by the instruction (i.e., “No” in operation 310), the method 300 returns to operation 302 of displaying the screensaver and operation 304 of monitoring the patient, which as described above, can occur simultaneously. When the location of the display device 200 is an authorized location defined by the instruction (i.e., “Yes” in operation 310), the method 300 proceeds to an operation 312 of determining whether a user having an authorized role is detected within a threshold distance of the display device 200.

Operation 312 can include using proximity detection technology to detect whether a user is within the a threshold distance of the display device 200. Operation 312 can include using the antenna 218 of the display device 200 to detect whether a user is within the a threshold distance of the display device 200 and to further identify the user. As described above, the antenna 218 can be used to detect wireless signals from objects worn or carried by a user. The wireless signal from each object is unique such that the wireless signals detected by the antenna 218 can be used to determine whether the user is within the threshold distance of the display device 200, and to further identify the role of the user such as whether the user is a physician, a registered nurse (RN), a licensed practical nurse (LPN), or other type of healthcare professional. The threshold distance used in operation 312 can be based on a proximity to the patient support apparatus 100 (i.e., bedside), a reading distance away from the display device 200, or another metric that protects the confidentiality of the patient's health data.

In alternative examples, operation 312 can include analyzing the visual data captured by the camera 50 to detect whether a user is within the threshold distance of the display device 200 and to also identify a role of the user. For example, the visual data can be analyzed to determine whether the user is within the threshold distance of the display device 200, and to read machine-readable data, a name, or other identification on an object worn by the user such as a badge, lanyard, name tag, and the like to determine the role of the user.

As illustrated in FIG. 3, when a user having an authorized role is not detected within the threshold distance of the display device 200 (i.e., “No” in operation 312), the method 300 returns to operation 302 of displaying the screensaver and operation 304 of monitoring the patient, which as described above, can occur simultaneously. When a user having an authorized role is detected within the threshold distance of the display device 200 (i.e., “Yes” in operation 312), the method 300 proceeds to an operation 314 of dynamically modifying the display screen 212 based on the location of the display device 200 and the role of the user detected as being within the threshold distance of the display device 200.

Operation 314 can include displaying one or more events detected in operation 306 based on the instruction generated in operation 308. For example, the one or more events are displayed on the display screen 212 of the display device 200 when the location of the display device 200 is included in the one or more authorized locations for displaying the one or more events and the user within the threshold distance of the display device 200 has a role that is included in the one or more authorized roles for viewing the one or more events.

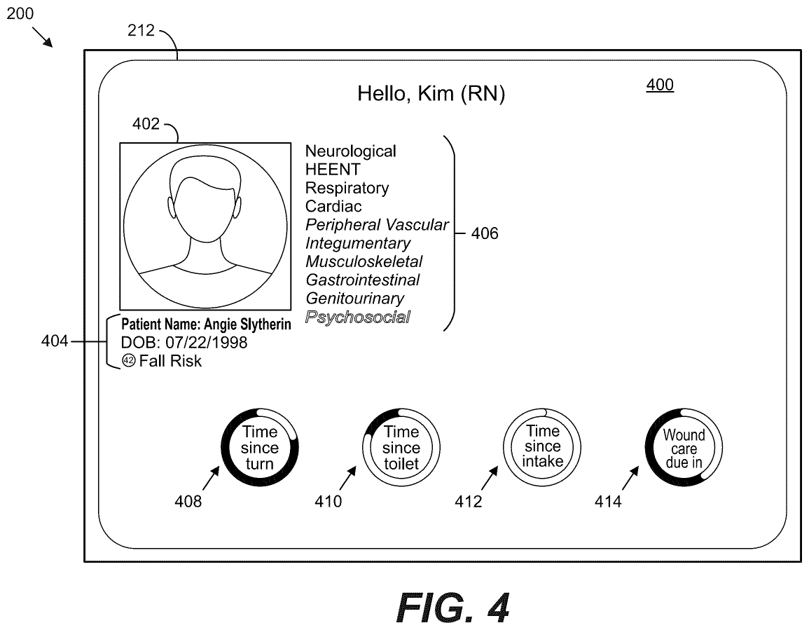

FIG. 4 illustrates an example of a graphical user interface 400 displayed on the display screen 212 of the display device 200 in accordance with operation 314 of the method 300. In this illustrative example, the graphical user interface 400 is displayed when a user having a role of a registered nurse (RN) is detected as being within the threshold distance of the display device 200, and the display device 200 is determined as being in an authorized location.

In the example shown in FIG. 4, the graphical user interface 400 includes relevant patient information such as an image 402 of the patient, and biographical data 404 of the patient such as their name, date of birth, and whether they have any elevated risks (e.g., fall risk). In some examples, the image 402 of the patient is captured by the camera 50.

The graphical user interface 400 further includes a customized task list 406 that is dynamically displayed based on the role of the registered nurse. The customized task list 406 includes tasks for the registered nurse to complete while they are bedside (i.e., within the threshold distance of the display device 200 mounted on the patient support apparatus 100).

In some examples when the display device 200 detects multiple users within the threshold distance of the display device 200, the display device 200 displays a customized task list that combines the task lists of the multiple users. In further examples, the display device 200 can prioritize tasks in the customized task list for a particular user when multiple users are detected in the vicinity of the patient support apparatus 100.

The graphical user interface 400 includes one or more timers 408-414 each associated with a clinically relevant event. The timers 408-414 are dynamically displayed based on the role of the registered nurse such that when a different user having a different role is detected as being within the threshold distance of the display device 200, different timers associated with different types of clinically relevant events can be displayed on the graphical user interface 400. Also, the timers 408-414 are dynamically displayed based on the condition of the patient.

In the example of FIG. 4, a first timer 408 shows a time since the patient last turned on the patient support apparatus 100, a second timer 410 shows a time since the patient last toileted, a third timer 412 shows a time since the patient last took in food and/or beverage, and a fourth timer 414 shows a time since a wound of the patient was last cared for. Additional types of timers or fewer types of timers can be displayed on the display screen 212 based on the role of the user who is detected as being within the threshold distance of the display device 200.

When a timer reaches a maximum time allotment (i.e., when the circle is completely full), an action associated with the timer needs to be completed by the authorized user. For example, FIG. 4 illustrates the third timer 412 as having reached a maximum time allotment such that the patient should be given food and beverage for intake. In the example of FIG. 4, the first, second, and fourth timers 408, 410, 414 have not reached their maximum time allotment such that the registered nurse does not need to take immediate action to turn the patient, to help the patient exit the patient support apparatus 100 to toilet, or to provide wound care.

The graphical user interface 400 allows the authorized user (e.g., registered nurse) to configure the graphical user interface 400 as desired such as to display different tiles and to add and/or remove certain information. Also, the information displayed on the graphical user interface 400 can be configured to be displayed in a graphical form to better enable trending.

The graphical user interface 400 allows an authorized user such as a registered nurse to add non-medical notes about the patient (e.g., the names of their children) that the registered nurse may want to remember when the registered nurse revisits the patient. Also, the graphical user interface 400 allows the registered nurse to add reminders to do some action at a later date such as when the registered nurse revisits the patient. When an authorized user such as a technician is detected as being within the threshold distance of the display device 200, the display device 200 displays servicing notifications for the display device 200 or other devices connected to the display device 200 and/or the network 20. Accordingly, the graphical user interface 400 can be configured to display non-chartable data on the display screen 212 based on an identity of the user detected within the threshold distance of the display device 200.

The notes, reminders, preferences, and other non-chartable information is uniquely displayed on the display device 200 based on detection of the identity of the user. For example, a set of notes, reminders, preferences, and other non-chartable information can be displayed on the display device when a particular user approaches the display device 200, while another set of notes, reminders, preferences, and other non-chartable information is displayed when another user approaches the display device 200. In this manner, the display device 200 can efficiently display the notes, reminders, and preferences for a plurality of different users.

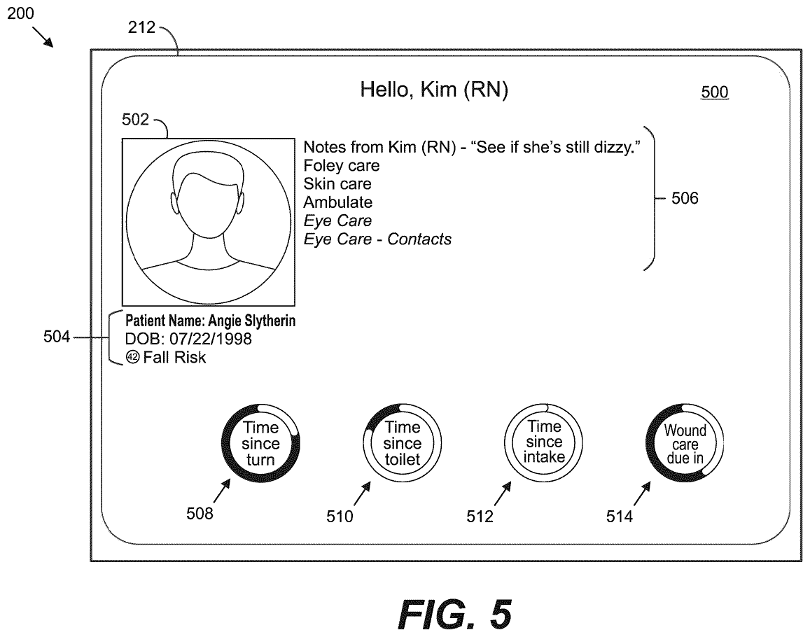

FIG. 5 illustrates another example of a graphical user interface 500 displayed on the display screen 212 of the display device 200 in accordance with operation 314 of the method 300. In this example, the graphical user interface 500 is displayed when a user having a role of a licensed practical nurse (LPN) is detected as being within the threshold distance of the display device 200 and the display device 200 is determined as being in an authorized location.

The graphical user interface 500 includes relevant patient information such as an image 502 of the patient, and biographical data 504 of the patient such as their name, date of birth, and whether they have any elevated risks (e.g., fall risk). The graphical user interface 500 further includes a customized task list 506 based on the role of the licensed practical nurse. The customized task list 506 includes tasks for the licensed practical nurse to complete while they are bedside (i.e., within the threshold distance of the display device 200 mounted on the patient support apparatus 100). The customized task list 506 for the licensed practical nurse differs from the customized task list 406 for the registered nurse shown in FIG. 4. The graphical user interface 500 further includes one or more timers 508-514 each associated with a clinical event relevant to the role of the licensed practical nurse. As discussed above, the timers are dynamically displayed based on the role of the authorized user and the condition of the patient.

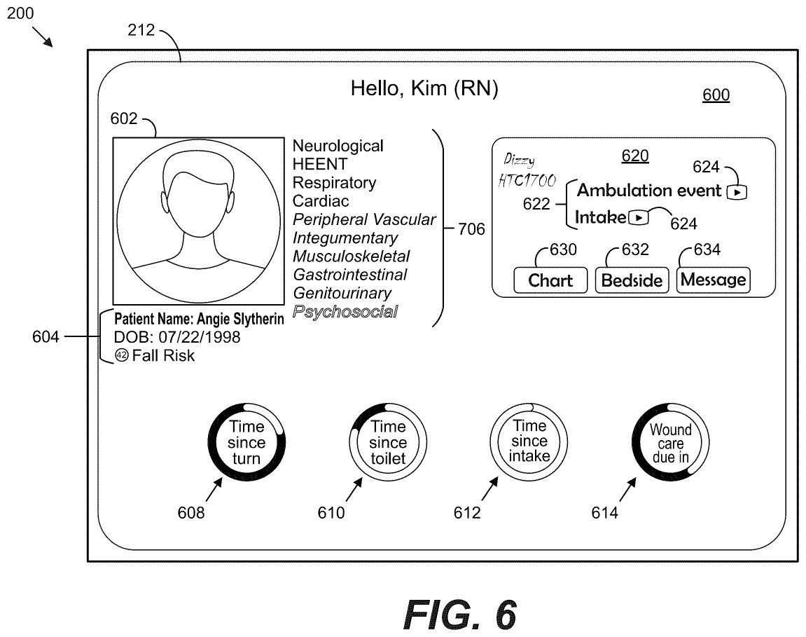

FIG. 6 illustrates another example of a graphical user interface 600 displayed on the display screen 212 of the display device 200 in accordance with operation 314 of the method 300. In this illustrative example, the graphical user interface 600 is displayed when a user having a role of a registered nurse (RN) is detected as being within the threshold distance of the display device 200, and the display device 200 is determined as being in an authorized location.

Like the example of the graphical user interface 400 of FIG. 4, the graphical user interface 600 includes relevant patient information such as an image 602 of the patient captured by the camera 50, and biographical data 604 of the patient such as their name, date of birth, and whether they have any elevated risks (e.g., fall risk), a customized task list 606 based on the role of the registered nurse, and one or more timers 608-614 each associated with a clinical event relevant to the role of the registered nurse and the condition of the patient.

The graphical user interface 600 further includes a staging area 620 that displays a list of events 622 detected in accordance with operation 306 of the method 300. The events are displayed in the staging area 620 until they are confirmed or discarded by an authorized user. The staging area 620 can also allow the authorized user to enter quick notes.

The staging area 620 displays the list of events 622 in accordance with the instruction generated in operation 308 such that the list of events 622 are displayed based on the location of the display device 200 and the role of the user who is detected as being within the threshold distance of the display device 200. Accordingly, the list of events 622 may vary depending on the location of the display device 200 and the role of the user who is within the threshold distance of the display device. The list of events 622 is obscured when the location of the display device 200 is not in one or more authorized locations defined by the instruction or when a user having an authorized role is not detected within a threshold distance of the display device 200.

In the example shown in FIG. 6, each event in the list of events 622 includes a playback icon 624 that when selected causes a video captured by the camera 50 to be played back on the display screen 212 of the display device. The video can be captured by the camera 50 while the monitoring of the patient is performed in operation 304 of the method 300.

The user having the authorized role can drag each event in the list of events 622 to one or more options displayed inside the staging area 620 to perform an action on the event. For example, the user can drag an event in the list of events 622 to one or more options displayed in the staging area 620 by swiping the user's finger or a stylus on the display screen 212.

The user can drag an event to a charting option 630 to have the event added to the EHR 42 of the patient. In such examples, the event is stored as chartable information.

The user can drag an event to a bedside option 632 to have the event saved to a local memory on the display device 200 or elsewhere such that the event can be accessed for display during a subsequent visit to the patient. In some examples, the event can be stored as non-chartable information. The bedside option 632 allows non-chartable data to be saved for future access and/or display to remind the user the next time the user visits the patient's bedside.

The user can drag an event to a message option 634 to have the event sent to one or more other users. For example, dragging an event to the message option 634 causes the display device 200 to communicate with the nurse call system 30 to message the event to one or more mobile devices 32 used by one or more other users of the system 10 to notify the one or more other users about the event. In some examples, the other users are notified based on their role.

While not shown in FIG. 6, the staging area 620 can include an option to discard the events. Also, based on the events displayed in the staging area 620, the user can reset one or more of the timers 608-614. For example, when an event shows that the patient took in food and beverage, the use can reset the third timer 612 for food and beverage intake.

Also, the user can utter a voice command to perform one or more actions on the events in the list of events 622 without the user having to physically touch the display screen 212. For example, the user can utter voice commands such as “Chart the ambulation event” or “Discard the intake event.” The microphone/speaker unit 214 detects the voice command and the LLM 210 analyzes the voice command to determine an action for the event such as to add the event as chartable information to the EHR 42 of the patient, store the event as non-chartable information, message the event to one or more other users, or discard the event.

In further examples, a sensor of the display device 200 can detect gestures by the user to perform actions on the events in the list of events 622 without the user having to physically touch the display screen 212. The gestures can be analyzed by the display device 200 to determine inputs for performing one or more actions on the display device 200 such as to add an event as chartable information to the EHR 42 of the patient, store an event as non-chartable information, or to message an event to one or more other users.

Referring back to FIG. 3, the method 300 can further include an operation 316 of monitoring an interaction between the patient and the user within the threshold distance of the display device 200. Operation 316 can include receiving audio data from the microphone/speaker unit 214 and visual data captured by the camera 50 while the user interacts with the patient.

The method 300 can further include an operation 318 of detecting one or more additional events based on the monitoring of the interaction between the patient and the user within the threshold distance of the display device 200. The additional events detected in operation 318 can include both chartable information and non-chartable information.

Operation 318 can include using the LLM 210 to analyze the audio data from the microphone/speaker unit 214 to detect the one or more additional events. The LLM 210 can analyze the audio data to detect chartable information such as one or more symptoms that are uttered or expressed by the patient while conversing with the user within the threshold distance of the display device 200. In another example, the LLM 210 can analyze the audio data to detect non-chartable information such as the names of the spouse, children, or other family members of the patient that the user may want to remember for developing a good rapport with the patient.

Operation 318 can also include analyzing the visual data captured by the camera 50 to detect the one or more additional events such as chartable actions performed by the authorized user while interacting with the patient such as when the authorized user turns the patient, helps the patient exit the patient support apparatus 100 to toilet, and provides wound care.

Operation 318 can also include receiving one or more direct inputs from an authorized user such as a clinician. For example, the authorized user can use the stylus to write notes on the display screen 212 or can type notes using a keyboard on the display screen. The authorized user can also select one or more input options on the display screen such as the capture of one or more vital signs by one or more sensors monitoring the patient.

The method 300 can further include an operation 320 of generating an instruction for displaying the one or more additional events detected in operation 318. The instruction is generated to have a data structure to facilitate storage and display of the event.

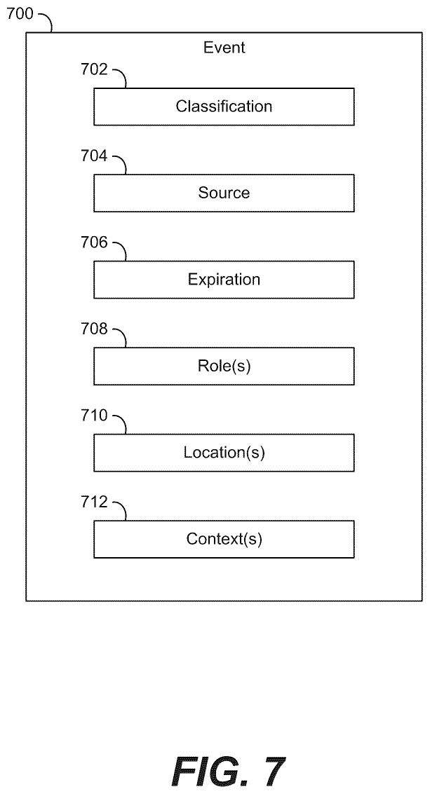

FIG. 7 schematically illustrates an example of a data structure 700 for displaying an event on the display device 200 in accordance with the operations of the method 300. The data structure 700 can include a classification 702 such as whether the event is chartable information or non-chartable information. Examples of chartable information can include one or more symptoms verbally uttered or expressed by the patient, and one or more actions performed by the patient or another user such as turning on the patient support apparatus 100, exiting the patient support apparatus 100 to toilet, providing wound care to the patient, and other actions. When the event is classified as chartable information, the event can be stored in the EHR 42 of the patient upon confirmation from an authorized user.

Examples of non-chartable information can include the names of the patient's family members, the patient's food/beverage preferences, how the patient likes to take their medications, and other information that can be used to build a rapport with the patient. When the event is classified as non-chartable information, the event can be stored locally on the display device 200 or elsewhere upon confirmation from an authorized user. When saved locally on the display device 200 or elsewhere, the non-chartable information can be displayed as a reminder when the authorized user revisits the patient such as during a follow-up visit.

The data structure 700 can include a source 704 of the event such as whether the event is detected from the EHR 42 of the patient, from ambient listening or watching of the patient and their interactions with other users by the camera 50 and the microphone/speaker unit 214, or from direct input from an authorized user such as a clinician.

In regards to the ambient listening, the LLM 210 can be used to analyze the audio data captured by the microphone/speaker unit 214 to identify the event such as a symptom that is verbally uttered by the patient. Additional artificial intelligence and computer vision technology can be used to analyze the visual data captured by the camera 50 to identify the event.

The direct input from the authorized user can be received on the display screen 212 such as a written note that is either handwritten or typed on the display screen 212 or a value, parameter, descriptor, or other input entered into a field displayed on the display screen 212. The direct input from the authorized user can also include verbal commands that are recorded by the microphone/speaker unit 214 and processed by the LLM 210.

The data structure 700 can include an expiration 706 for displaying the event on the display device 200. For example, the event can be valid for display for X hours or days. Also, the display of the event can be contingent upon another event occurring or not occurring.

The data structure 700 further includes one or more authorized roles 708 for displaying the event on the display device 200. For example, the event can be displayed when a user such as a physician or registered nurse is detected as being within the threshold distance of the display device 200, and the event is not displayed when another user such as a licensed practical nurse is detected within the threshold distance of the display device 200.

The data structure 700 further includes one or more locations 710 for displaying the event on the display device 200. As an illustrative example, the event can be displayed when the display device 200 is detected as being in a designated patient room, but is not displayed when the display device 200 is detected as being outside of the designated patient room such as when in a hallway of the healthcare facility such as when the patient support apparatus 100 is being transferred from the designated patient room to another area in the healthcare facility.

The data structure 700 can include one or more contexts 712 for displaying the event on the display device 200 such as when the LLM 210 detects a topic being discussed between the patient and an authorized user within the threshold distance of the display device 200 based on the audio data captured by the microphone/speaker unit 214. When the event is relevant to the topic being discussed between the patient and an authorized user, the event is displayed on the display device 200 to facilitate the dialogue between the patient and the authorized user.

Referring back to FIG. 3, the method 300 can further include an operation 322 of displaying the one or more additional events based on the instruction generated in operation 320. For example, the one or more additional events can be displayed on the display screen 212 of the display device 200 based on the components of the data structure 700 such as the classification 702, the source 704, the expiration 706, the one or more authorized roles 708, the one or more locations 710, and the one or more contexts 712. The one or more additional events are displayed on the display screen 212 while the authorized user remains within the threshold distance of the display device 200. When the authorized user moves away from the display device 200 such that they are no longer within the threshold distance of the display device 200, the screensaver is displayed on the display screen 212 to obscure or hide the one or more additional events.

The method 300 can further include an operation 324 of receiving a confirmation from the authorized user to store the one or more additional events. The confirmation can be received via a voice instruction detected from the authorized user by the LLM 210 analyzing audio data captured by the microphone/speaker unit 214. Alternatively, the confirmation can be received via a hand gesture detected by a sensor of the display device 200, or via a swipe detected on the display screen 212 of the display device 200. The confirmation can be part of information staging where the events detected by the display device 200 can have multiple states that can vary based on where or how the event is detected. For example, when the source of the event is automatic detection, the information staging can include a state where the event needs to be confirmed by an authorized user in order to perform an action on the event such as to store the event in the EHR as chartable information, or to message the event to one or more other users.

Upon receipt of the confirmation, the method 300 includes an operation 326 of performing one or more actions on the event displayed on the display device 200. For example, the one or more actions performed on the event can include having the event added to the EHR 42 of the patient when the event includes chartable information, having the event saved to a local memory on the display device 200 or elsewhere when the event includes non-chartable information, messaging the event to one or more other authorized users by using the nurse call system 30, or checking off the event from the task list of an authorized user. Accordingly, the display device 200 acts as an ancillary display and/or hands-free interface of other medical devices such as the patient support apparatus 100, patient monitoring devices, and the like.

In view of the foregoing, each event detected by the display device 200 is stored having the data structure 700 for determining when and where to display the event on the display screen 212 including for users, locations, and contexts where the information of the event will be most efficiently and effectively used. Accordingly, the display device 200 is aware of an identity of the patient, a presence of one or more authorized users, and a location of the display device 200 to dynamically determine which information to display on the display screen 212.

The information displayed on the display device 200 can be acquired from various sources including the EHR 42 of the patient, patient-clinician interactions where the display device 200 is located, and clinician direct input on the display device 200. Artificial intelligence and/or computer vision technology can be used to create a suggested piece of information for storage and display on the display device 200, and when paired with an authorized user's confirmation, which can be either hands-free or just a simple swipe on the display screen 212, allows the information to be viewed without any further action from the user.

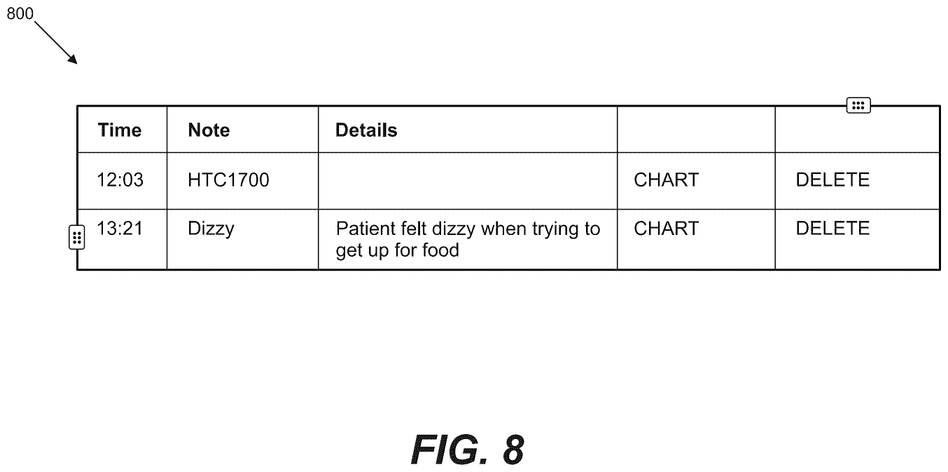

FIG. 8 schematically illustrates an example of data entries 800 that can be generated by the display device 200 based on the inputs and/or commands received from an authorized user. The data entries 800 can be stored in the EHR 42 of the patient, locally on the display device 200, or elsewhere. As shown in FIG. 8, more than the information displayed on the display device 200 is stored in the backend. For example, the data entries can include a time stamp of when information is entered by the user and a short note such as “HTC1700” which is a reminder that hematocrit level (i.e., a measurement of the percentage of red blood cells in blood) needs to be measured at time 1700 which is non-chartable information that reminds a registered nurse to draw blood and check the HTC result which can be part of a task list for the registered nurse. The data entries 800 can further include more detailed information such as “Patient felt dizzy when trying to get up for food” for a short note “dizzy.” The data entries 800 can further include an instruction such as whether to chart or not a piece of information, or to delete the information. The data entries 800 ensure that an event is populated in an appropriate area of a graphical user interface displayed on the display screen 212 of the display device 200.

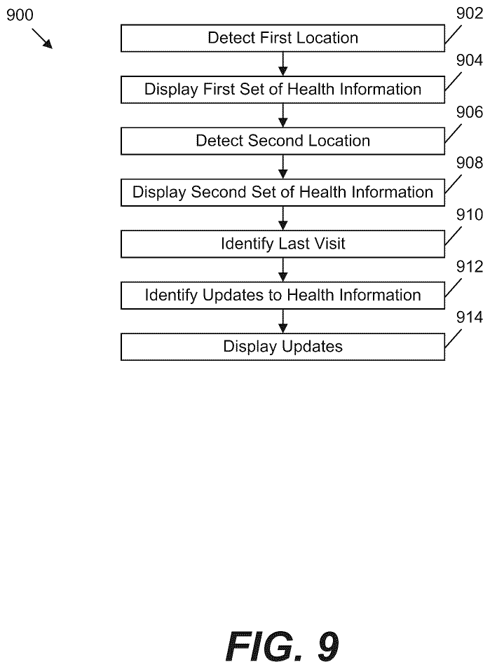

FIG. 9 schematically illustrates an example of a method 900 of dynamically displaying health information. The method 900 alters display of health information to mitigate cognitive overload that can be experienced by healthcare professionals by automatically adjusting the display of the health information based on the location of a healthcare professional such that different health information is displayed when the healthcare professional is bedside versus when in a hallway or an office of a healthcare facility.

The method 900 can be performed on the display device 200 such as when the display device 200 is removed from the patient support apparatus 100 and is being carried by an authorized user as the authorized user walks around the healthcare facility.

Alternatively, the method 900 can be performed on a mobile device 32 carried by a healthcare professional such as a physician, a registered nurse, or other type of caregiver. The mobile device 32 is carried by the healthcare professional while on shift in the healthcare facility. The mobile device 32 can share similar hardware with the display device 200 such as a computing device having at least one processing device and at least one memory device that stores software instructions that, when executed by the at least one processing device, cause the at least one processing device to perform the operations of the method 900. The mobile device 32 can further include a display screen, a microphone/speaker unit, a camera, an antenna, a network interface, a location tracker, and a rechargeable battery. Examples of the mobile device 32 can include smartphone, tablet computers, and other types of portable computing devices.

As shown in FIG. 9, the method 900 includes an operation 902 of detecting a first location of the display screen 212 of the display device 200, or alternatively, the display screen of the mobile device 32. The location of the display screen can be detected by using a location tracker (such as the location tracker 222 shown in FIG. 2) that tracks a location of the display screen by utilizing Bluetooth Low Energy (BLE) to broadcast a Bluetooth signal that is detected by other devices connected to the network 20 that are proximate to the display screen. The location of the display screen is determined based on the location of the other devices that are connected to the network 20. In further examples, the location tracker can utilize additional technologies for determining the location of the display screen such as GPS technology. In yet further examples, operation 902 can include detecting a first location of the display screen based on location tracking performed by another electronic item carried by the healthcare professional.

In further examples, operation 902 can include detecting a first location of the display screen based on the location of another device carried by the healthcare professional such as a wireless tag attached to an item worn by the healthcare professional such as a lanyard, name tag, and the like. The wireless tag emits a wireless signal (e.g., a radio signal, a Bluetooth® signal, and the like) that is detected by a real-time location system (RTLS) that includes readers (also known as anchors) that are positioned throughout the healthcare facility.

The method 900 includes an operation 904 of displaying a first set of health information on the display screen based on the first location. The first set of health information has a first specificity. As used herein, specificity means granularity of information displayed on the display screen based on the location of the display screen. As an illustrative example, when the first location is a hallway in a wing of the healthcare facility, the first specificity causes the health information displayed on the display screen to include health information for a plurality of patients that are assigned to rooms connected to the hallway. In some examples, the health information for the plurality of patients is prioritized based on risk severity of the plurality of patients. For example, operation 904 includes displaying health information only for patients who are deemed high risk, while health information for low risk patients is not displayed. Additionally, or alternatively, operation 904 can include displaying health information only for patients who have experienced a status change within a predefined window of time, while health information for patients who have not experienced a status change is not displayed.

The method 900 includes an operation 906 of detecting a second location of the display screen 212 of the display device 200, or alternatively, the display screen of the mobile device 32. Operation 906 can include detecting the second location of the display screen using the same technique used in operation 902 for detecting the first location of the display screen. In this illustrative example, the second location of the display screen detected in operation 906 differs from the first location of the display screen detected in operation 902.

The method 900 includes an operation 908 of displaying a second set of health information on the display screen based on the second location. The second set of health information has a second specificity that differs from the first specificity. As an illustrative example, the second location can include a patient room such that the second specificity displays health information for a single patient in closest proximity to the display screen. In such example, the second specificity is higher than the first specificity because the health information for a single patient is more granular than the health information for a plurality of patients.

In another illustrative example, the second location can include a command center such that the second specificity displays health information according to a command center mode. The sets of health information and their associated specificities are configurable such as by an administrator to provide customized dynamic views of the health information as desired.

In some examples, the first and second sets of health information are customized based on a role of the healthcare professional who is operating the mobile device 32 or the display device 200. For example, the first and second sets of health information can display different information based on whether the healthcare professional is a physician, registered nurse (RN), licensed practical nurse (LPN), or other type of healthcare professional.

In some examples, the method 900 can include an operation 910 of identifying a last visit to the single patient, an operation 912 of determining updates to the health information of the single patient since the last visit, and an operation 914 of displaying the updates to the health information since the last visit of the single patient. In this manner, the second set of health information having the second specificity is prioritized to display the updates to the health information since the healthcare professional's last visit to the room of the single patient.

In view of the foregoing, the method 900 displays the most relevant health information mapped to the time, location and current clinical mode of use. Also, the method 900 removes the need for healthcare professionals to fidget with their mobile devices to manually adjust views of voluminous health information while they are caring for their patients.

The various embodiments described above are provided by way of illustration only and should not be construed to be limiting in any way. Various modifications can be made to the embodiments described above without departing from the true spirit and scope of the disclosure.

What is claimed is:

1. A system for capturing and displaying health information, the system comprising:
a display device including:
a housing;
a display screen supported on the housing;
at least one processing device communicatively connected to the display screen; and
at least one memory device storing software instructions that, when executed by the at least one processing device, cause the at least one processing device to:
receive data from one or more devices monitoring a patient;
detect an event based on the data received from the one or more devices;
generate an instruction for displaying the event, the instruction including an identification of one or more authorized roles for viewing the event and an identification of one or more authorized locations for displaying the event;
determine a location of the display device;
detect a user within a threshold distance of the display device; and
display the event on the display screen when the location of the display device is included in the one or more authorized locations for displaying the event and a role of the user within the threshold distance of the display device is included in the one or more authorized roles for viewing the event.

2. The system of claim 1, wherein the instructions, when executed by the at least one processing device, further cause the at least one processing device to:
display a screensaver on the display screen when the location of the display device is not in the one or more authorized locations for displaying the event or when the role of the user is not included in the one or more authorized roles for viewing the event.

3. The system of claim 1, wherein the one or more devices include a camera capturing visual data of the patient, and wherein the event is detected based on an analysis of the visual data.

4. The system of claim 1, wherein the one or more devices include sensors for capturing measurements of one or more physiological variables of the patient, and wherein the event is detected based on the measurements of one or more physiological variables.

5. The system of claim 1, wherein the one or more devices include a microphone; and
wherein the instructions, when executed by the at least one processing device, further cause the at least one processing device to:
use the microphone to capture a recording of an interaction by the user with the patient and the display device;
detect a second event based on the recording of the interaction;
display the second event on the display screen while the user remains within the threshold distance of the display device; and
receive a confirmation from the user to store the second event.

6. The system of claim 5, wherein the instructions, when executed by the at least one processing device, further cause the at least one processing device to:
generate a second instruction for displaying the second event, the second instruction including a classification of whether the second event is chartable or non-chartable, an identification of a source of the second event, an expiration for displaying the second event, one or more authorized roles for displaying the second event, one or more authorized locations for displaying the second event, and one or more relevant contexts for displaying the second event.

7. The system of claim 5, wherein the second event is stored as chartable information in an electronic health record.

8. The system of claim 5, wherein the second event is stored as non-chartable information for display when the user is within the threshold distance of the display device.

9. The system of claim 5, wherein the confirmation is received via a voice instruction of the user, a hand gesture of the user, or a swipe on the display screen.

10. The system of claim 1, further comprising:
a patient support apparatus having a frame configured to support a mattress, and wherein the frame includes a docking station for the display device.

11. The system of claim 10, wherein the instructions, when executed by the at least one processing device, further cause the at least one processing device to:
trigger an alarm when the display device is moved beyond the threshold distance away from the patient support apparatus.

12. The system of claim 1, further comprising:
a location tracker for tracking a location of the display device.

13. The system of claim 1, wherein the instructions, when executed by the at least one processing device, further cause the at least one processing device to:
display a task list and one or more timers on the display screen based on a role of the user detected within the threshold distance of the display device.

14. The system of claim 1, wherein the instructions, when executed by the at least one processing device, further cause the at least one processing device to:
display non-chartable data on the display screen based on an identity of the user detected within the threshold distance of the display device.

15. The system of claim 1, wherein the display device includes a camera for capturing one or more images of the patient.

16. A method of dynamically displaying health information, the method comprising:
detecting a first location of a display screen;
displaying a first set of health information on the display screen based on the first location, the first set of health information having a first specificity;
detecting a second location of the display screen, wherein the second location of the display screen differs from the first location of the display screen; and
displaying a second set of health information on the display screen based on the second location, the second set of health information having a second specificity, and wherein the second specificity differs from the first specificity.

17. The method of claim 16, wherein the first specificity displays health information for a plurality of patients, and wherein the health information for the plurality of patients is prioritized based on risk severity of the plurality of patients.

18. The method of claim 16, wherein the second specificity displays health information for a single patient in closest proximity to the display screen.

19. The method of claim 18, further comprising:
identifying a last visit to the single patient;
determining updates to the health information of the single patient since the last visit; and
displaying the updates to the health information since the last visit of the single patient.

20. The method of claim 16, wherein the first and second sets of health information are customized based on a role of a user of the device.
